(19) United States
(12) Plant Patent Application Publication
(10) Pub. No.: US 2013/0269071 P1
(43) Pub. Date: Oct. 10, 2013

(54) Grapevine 'SHEEGENE-16'

(50) ;

(71) Applicant: Timothy P. Sheehan, Vancouver, WA (US)

(72) Inventor: Timothy P. Sheehan, Vancouver, WA (US)

(21) Appl. No.: 13/506,258

(22) Filed: Apr. 6, 2012

(51) Int. Cl. A01H 5/00

(52) U.S. Cl. PLT/205

(57) ABSTRACT

A new and distinct variety of grapevine characterized by the production of large, red-colored seeded grapes that mature in early September, approximately six to eight weeks later than ‘Flame Seedless’ (unpatented) when grown in the San Joaquin Valley of Central California. The grapes of this new variety are produced on strong woody stems and branches and are well adapted to commercial handling.

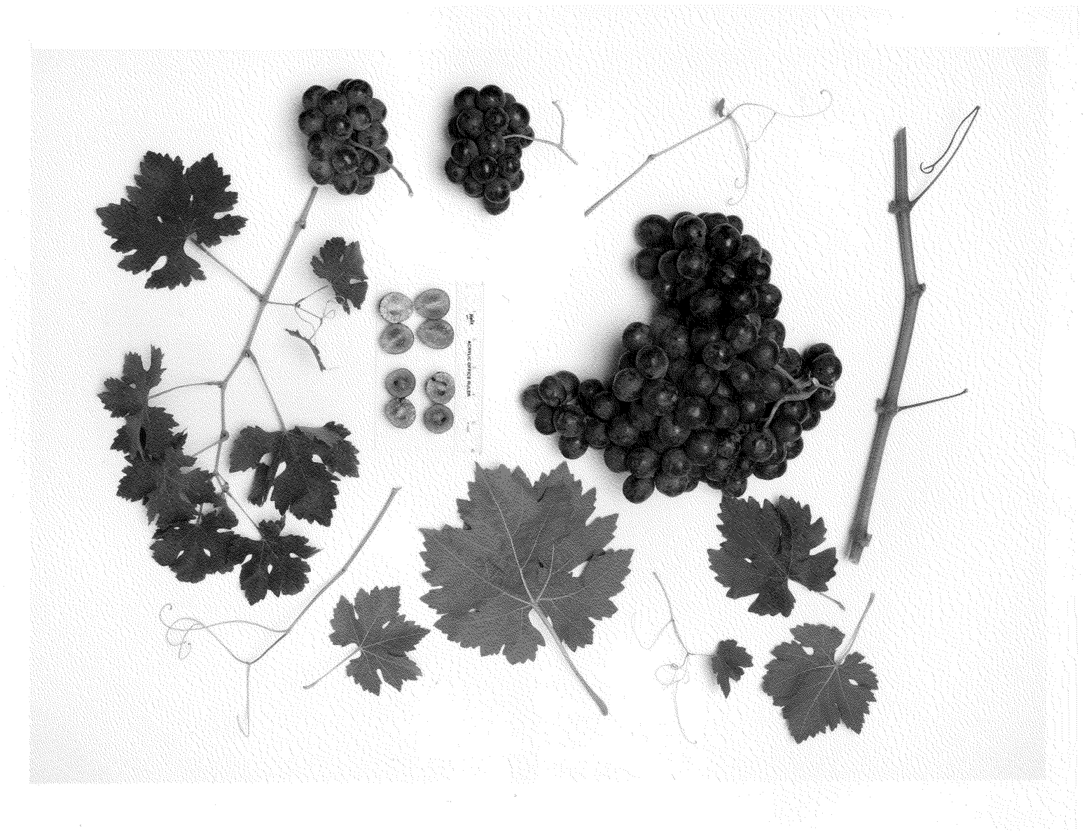

### CROSS REFERENCE TO RELATED APPLICATIONS

The claimed plant is not subject of a related application.

### STATEMENT OF ANY FEDERALLY-SPONSORED RESEARCH AND DEVELOPMENT

The claimed plant is not subject of Federally-sponsored research or development.

### LATIN NAME OF THE GENIUS AND SPECIES OF THE PLANT CLAIMED

The claimed plant relates to a new and distinct variety of Vitis vinifera to be known as ‘Sheegene-16’.

### VARIETY DENOMINATION

The new variety of Vitis vinifera is the result of hybridization of ‘R5V38-44’ (unpatented), the pollen parent, and ‘Red Globe’ (U.S. Plant Pat. No. 4,787), the seed parent. The new variety was asexually propagated by Timothy P. Sheehan in the Spring of 2000 in a Vitis vinifera variety block located near East of Fowler, Calif. The new variety was planted as rooted cuttings in June of 2007 in a field located northwest of Delano, Calif. The new variety produces a large, red-colored seeded grape with very good flavor. The new variety has been shown to maintain its distinguishing characteristics through asexual propagation.

### BACKGROUND OF THE INVENTION

The present invention relates to a new and distinct variety of grapevine that produces a large, red-colored seeded grape that matures during early September when grown in the San Joaquin Valley of Central California. The new invention resembles ‘Flame Seedless’ (unpatented).

### SUMMARY OF THE INVENTION

The ‘Sheegene-16’ grapevine is characterized by producing a large, red-colored seeded grape that has very good flavor and it is productive and mature for harvesting and shipment in early September when grown in the San Joaquin Valley of Central California. The new variety can be compared to ‘Flame Seedless’ (unpatented) but the grapes produced by the new variety mature approximately six to eight weeks later, among other distinguishing characteristics.

### BRIEF DESCRIPTION OF THE DRAWINGS

The accompanying drawing is a color photograph that shows a branch displaying nodes along the top portion of the photograph, a cluster of mature grapes is located directly below with several immature leaves shown to the right of the cluster, directly below the immature leaves is a mature leaf and an immature leaf, two small clusters of mature grapes and four grapes cut in half displaying the color of the flesh and the shape of the berries are shown in lower-left center of the photograph, and at the bottom of the photograph is an immature branch displaying leaves and tendrils.

### DETAILED BOTANICAL DESCRIPTION

Referring more to the horticultural description of the new and distinct variety of grapevine, the following has been observed under the ecological conditions prevailing at the origin vineyard located near Delano, Calif., in the San Joaquin Valley of Central California. All major color descriptions are by reference to the Dictionary of Color by Maerz & Paul, First Edition, published in 1930. Common colors names are also used in several instances.
Vine:
   Size.—Medium to large.
   Vigor.—Very good.
   Chilling requirements.—Normal for grapevines in the lower San Joaquin Valley of Central California.
   Figure.—With wide cordons forming a “T” shape.
   Productive capacity.—Very good.
   Regularity of bearing.—Regular.
Trunk:
   Size.—Medium to large; 2 inches [5.08 cm] in circumference, 2.4 inches [6.10 cm] above ground.
   Surface texture.—Rough, shaggy.
   Color of bark.—Pl .7 H1 Casserole Rosite Gingerspice Eskimo+.
   Mature cane color.—Pl .7 H9 Liver Brown Autumn Oak−.
   Nodes.—Eight nodes on cane, cane is 21 inches [53.34 cm] long.
   Length between nodes.—3½ to 6 inches [8.89 to 15.24 cm].
   Shoot length.—6 inches [15.24 cm].
   Shoot shape.—Slender.
   Shoot contour.—Droopy.
   Tendrils number/location.—Single at node.
   Tendrils length.—2⅞ inches [7.30 cm].
   Tendrils form.—Bifurcate.
   Bud shape.—Triangular.
   Bud size.—Small; 0.078 inches [2 mm].
Branches:
   Size.—Medium.
   Cordons.—25 inches (63.50 cm) on one side of trunk; 22½ inches (57.15 cm) on the other side of trunk.
   Surface texture.—Rough.
   Color (One year or older branches).—Pl .6 L5 Corinthian Pr.
   Color (Immature branches).—Pl .5 G4 Blush Rose (Upper side); Pl .19 E6 Calliste Green (Lower side).
Leaves:
   Size.—Large.
   Density.—Dense.
   Average length.—5½ to 7 inches [13.97 to 17.78 cm].
   Average width.—4½ to 8 inches [11.43 to 20.32 cm].
   Form.—Pentagonal.
   Texture (Upper surface).—Smooth.
   Texture (Lower surface).—Slightly glabrous.
   Leaf vein color.—Pl .20 K1 Russet gr.
   Margin form.—Undulate.
   Leaf margin.—Toothed.
   Leaf vein thickness.—0.08 inches [2 mm].
   Stem glands.—None.
   Petiole size.—Moderate.
   Petiole length.—3 inches [7.62 cm].
   Petiole color (Lower surface.—Pl .9 H7 Liverbrown+Autumn Oak.
   Petiole color (Upper surface).—Pl .17 L7 Viridine y.
   Leaf sinus form.—Upside down ‘U’ shape.
   Lobe (Average).—Four.
   Stipules.—None.
   Tooth size.—0.20 to 0.31 inches [5 to 8 mm] in width; 0.28 to 0.31 inches [7 to 8 mm] in length.
   Tooth number.—Four.
Inflorescence:
   Size.—5.51 to 6.69 inches [14 to 17 cm].
   Number per spur.—One to two.
   Number per vine.—Many; up to fifty-two clusters.
Flowers:
   Flower buds.—0.04 to 0.08 inches [1 to 2 mm].
   Flower buds surface.—Glabrous.
   Flower buds.—Generally heavy.
   Date of bloom.—May 7.
   Date of full bloom.—May 12 to 14.
   Size (Average).—0.20 inches [5 mm].
   Petals (Color).—Pl .7 C8 Mauve Taupe.
   Petals (Size).—0.16 inches [4 mm].
   Pistil color.—Pl .17 L8 Nova gr.
   Date of visible berry set.—May 12 to 14.
   Size of berries.—0.08 inches [2 mm].
Fruit:
   Solids.—18.2 Brix.
   Acids.—0.34.
   Sugar/acid ratio.—53.5.
   Juice pH.—3.97.
   Seeds.—Several.
   Seed number.—One to two.
   Capstem pedicel.—0.47 to 0.98 inches [12 to 25 mm].
   Berry weight.—53.9 grams (1.90 oz).
   Juice color.—Pl .7 J8 Dominge.
   Cluster size.—Large.
   Compactness.—Compact.
   Cluster average length (not including stem).—6.50 inches [16.51 cm].
   Cluster average diameter.—6.50 inches [16.51 cm].
   Cluster average weight.—1.19 lb. [539.13 grams].
   Compactness.—Compact.
   Cluster form.—Conical.
   Stem.—Generally large.
   Stem caliper.—3.5 inches [8.89 cm].
   Berry size.—Large.
   Berry form.—Ovate.
   Berry numbers (Average).—90 berries per bunch.
   Berry size (Average Dimension Along Longitudinal Axis).—1.00 Inches [25.33 mm].
Skin:
   Skin thickness.—Medium.
   Texture.—Semi-tough.
   Blush color.—Pl .18 L6 Love Bird.
   Ground color.—Pl .56 L12 Port Wine.
   Lenticels.—None.
Flesh:
   Flesh color.—Pl .18 L6 Love Bird.
   Juice production.—Good.
   Flavor.—Very good.
   Aroma.—Mild.
   Texture.—Firm.
   Ripening.—Even.
   Eating quality.—Very good.
   Resistance to disease.—Unknown.
   Harvesting.—Early September in the San Joaquin Valley of Central California.
   Shipping and handling qualities.—Very good.

Having thus described and illustrated our new variety of grapevine, we claim:

1. A new variety of grapevine to be known as ‘Sheegene-16’ and parts thereof, substantially as illustrated and described, characterized principally by its production of large, red-colored seeded grapes that mature six to eight weeks later in the season of ripening than ‘Flame Seedless’ (unpatented) which it resembles.
